(19) United States
(12) Patent Application Publication
(10) Pub. No.: US 2017/0209312 A1
(43) Pub. Date: Jul. 27, 2017

(54) ABSORBENT ARTICLES INCLUDING SILICONE

(71) Applicant: Covidien LP, Mansfield, MA (US)

(72) Inventors: Anand Kanchagar, Beaverton, OR (US); Carlos Gutierrez, Rancho Santa Margarita, CA (US)

(21) Appl. No.: 15/411,524

(22) Filed: Jan. 20, 2017

Related U.S. Application Data
(60) Provisional application No. 62/281,793, filed on Jan. 22, 2016.

(51) Int. Cl. A61F 13/02; A61F 13/00; A61F 13/53

(52) U.S. Cl. CPC A61F 13/0253; A61F 13/53; A61F 13/0203; A61F 13/0259; A61F 13/00987; A61F 2013/530481; A61F 2013/00263

(57) ABSTRACT

Example absorbent articles including an adherent member comprising a silicone, a substrate adjacent the adherent member, and an absorbent member disposed between the adherent member and the substrate are disclosed. The adherent member defines a plurality of perforations extending through a thickness of the adherent member. Example techniques for forming example articles are disclosed.

This application claims the benefit of U.S. Provisional Application No. 62/281,793, filed Jan. 22, 2016, which is incorporated by reference herein in its entirety.

### TECHNICAL FIELD

The present disclosure generally relates to absorbent articles, for example, wound dressings.

### BACKGROUND

Some absorbent articles such as wound dressings include an absorbent material capable of absorbing fluids such as wound exudates. Retention of fluids in contact with skin, for example, near wounds, may reduce the effectiveness of wound dressings in promoting wound healing. Retained fluids may also reduce adhesion between absorbent articles and skin, leading to premature peeling off of the absorbent articles from a skin surface of a patient.

### SUMMARY

In some examples described herein, an absorbent article comprises an adherent member including silicone, where the adherent member may be configured to contact a skin surface of a patient during use of the absorbent article. The absorbent article further includes a substrate, and an absorbent member disposed between the adherent member and the substrate. A portion of the substrate may be adjacent the adherent member in some examples. In some examples, the adherent member may define a plurality of perforations extending through a thickness of the adherent member.

One or more examples of the present disclosure pertain to an example technique of forming an absorbent article. The example technique includes securing an absorbent member to a substrate, and securing at least a portion of an adherent member including a silicone to at least a portion of the absorbent member. In some examples, the adherent member defines a plurality of perforations extending through a thickness of the adherent member.

Clause 1: An absorbent article comprising: an adherent member comprising silicone, wherein the adherent member defines a plurality of perforations extending through a thickness of the adherent member; a substrate adjacent the adherent member; and an absorbent member disposed between the adherent member and the substrate:

Clause 2: The absorbent article of clause 1, wherein the adherent member is configured to adhere to skin of a patient.

Clause 3: The absorbent article of clause 1 or 2, wherein the adherent member comprises a plurality of laminated layers.

Clause 4: The absorbent article of clause 3, wherein at least one layer of the plurality of laminated layers consists essentially of a silicone layer configured to contact skin of a patient and adhere the absorbent article to skin.

Clause 5: The absorbent article of clause 3, wherein at least one layer of the plurality of layers consists essentially of an adhesive layer adhering the absorbent member to the adherent member.

Clause 6: The absorbent article of any one of clauses 1 to 5, further comprising a release member disposed on the adherent member.

Clause 7: The absorbent article of clause 6, wherein the release member comprises a polycarbonate film.

Clause 8: The absorbent article of any one of clauses 1 to 7, wherein the absorbent member comprises a superabsorbent material.

Clause 9: The absorbent article of any one of clauses 1 to 8, wherein the substrate is moisture vapor permeable.

Clause 10: The absorbent article of clause 9, wherein the substrate has a moisture vapor transmission rate of at least about 500 grams per meters squared per day.

Clause 11. The absorbent article of clause 9, wherein the substrate comprises a laminated member.

Clause 12: The absorbent article of clause 11, wherein the laminated member comprises a nonwoven layer and a supporting film.

Clause 13: A wound dressing comprising the absorbent article of any one of clauses 1 to 12.

Clause 14: A method of forming an absorbent article, the method comprising: securing an absorbent member to a substrate; and securing at least a portion of an adherent member comprising silicone to at least a portion of the absorbent member, wherein the adherent member defines a plurality of perforations extending through a thickness of the adherent member.

Clause 15: The method of clause 14, further comprising securing at least another portion of the adherent member to at least a portion of the substrate.

Clause 16: The method of clause 14 or 15, further comprising applying a release member on the adherent member.

Clause 17: The method of any one of clauses 14 to 16, wherein the securing comprises one or more of heat bonding, adhesive bonding, static bonding, or corona treatment.

Clause 18: The method of any one of clauses 14 to 17, wherein the absorbent member comprises a superabsorbent material.

Clause 19: The method of any one of clauses 14 to 18, wherein the substrate is moisture vapor permeable.

Clause 20: The absorbent article of clause 19, wherein the substrate has a moisture vapor transmission rate of at least about 500 grams per meters squared per day.

### DESCRIPTION OF THE DRAWINGS

FIG. 1 is a schematic and conceptual illustration showing a plan view of an absorbent article.

FIG. 2 is a schematic and conceptual illustration showing cross-sectional view of the absorbent article of FIG. 1, further including a release member.

FIG. 3 is a schematic and conceptual illustration showing an exploded cross-sectional view of the absorbent article of FIG. 2.

FIG. 4 is a schematic and conceptual illustration showing a plan view of an example absorbent article including a release member.

The details of one or more aspects of the disclosure are set forth in the accompanying drawings and the description below. Other features, Objects, and advantages of the techniques described in this disclosure will be apparent from the description and drawings, and from the claims.

### DETAILED DESCRIPTION

In the present disclosure, the terms “proximal” and “distal” indicate relative proximity to a reference surface. For example, “proximal” indicates a location relatively nearer the reference surface, while “distal” indicates a location relatively farther from the reference surface. In some examples, the reference surface may include skin to which example absorbent articles according to the disclosure may be configured to be applied. Thus, a proximal surface may be a surface configured to face toward skin, while a distal surface may be a surface configured to face away from skin when an absorbent article is placed on a skin surface of a patient such that an absorbent member of the absorbent article absorbs substances from the patient, such as wound exudate.

In some examples, absorbent articles according to the disclosure include an adherent member including silicone, for example, a silicone adhesive, configured to contact a skin surface. In comparison to other kinds of adhesives, such as acrylic adhesives, silicone act as a relative gentle adhesive that may reduce discomfort at the skin surface to which the absorbent article is applied or secured using the adherent member. The adherent member may facilitate the absorbent article, for example, a wound dressing, to remain in place on a skin surface of a patient, and help prevent or reduce the likelihood of contaminations of the tissue or skin as a wound heals or as wound fluid dries. The silicone may be of medical grade. The adherent member may define perforations extending through a thickness of the adherent member that allow body fluids, for example, perspiration or wound exudates, to flow across the adherent member into an absorbent core of an absorbent member of the absorbent article.

In some examples, example absorbent articles according to the present disclosure may exhibit a high absorbent capacity, a high moisture vapor permeability, or any combination thereof. The high moisture vapor permeability may allow the absorbent article to breathe, which may increase patient comfort and facilitate wound healing if the absorbent article is used as a wound dressing. Wound dressings including example absorbent articles described herein may be used for treating heavily exuding wounds as well as acute wounds.

In some examples, absorbent articles according to the present disclosure may be relatively thin and conformable. For example, this may allow the absorbent article, for example, a wound dressing, to be placed on regions of a person's body that are at risk of pressure ulcers.

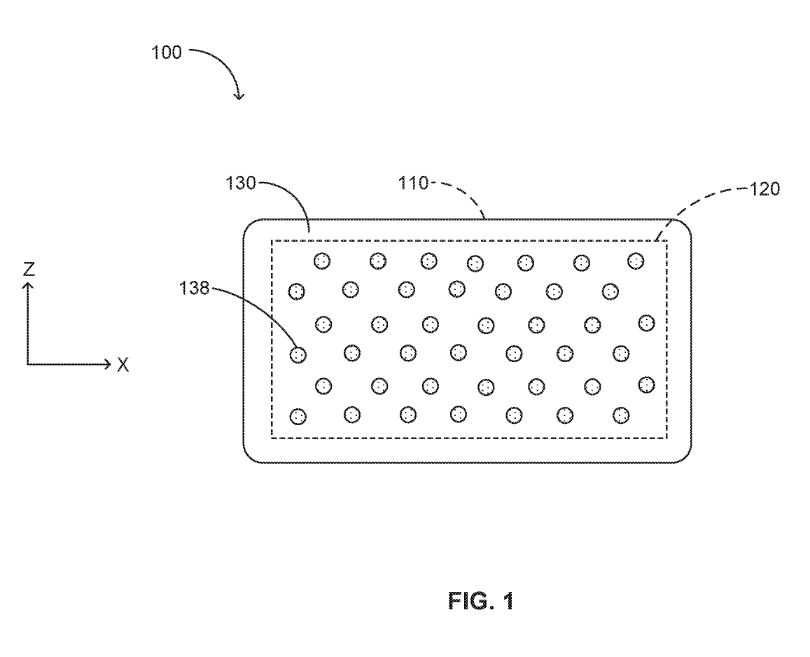

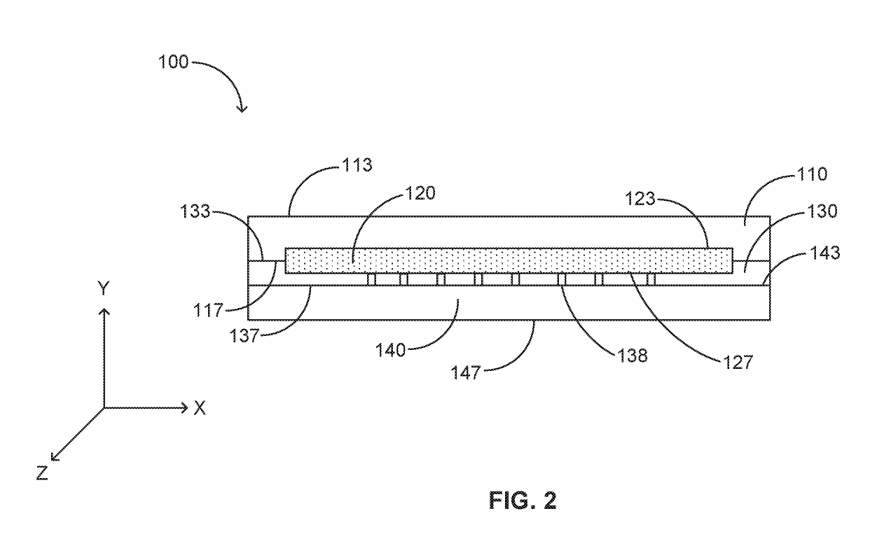

FIG. 1 is a schematic and conceptual illustration showing a plan view of an example absorbent article 100 according to the present disclosure. FIG. 2 is a schematic and conceptual illustration showing a cross-sectional view of absorbent article 100 of FIG. 1, further including a release member. Absorbent article 100 may be an article configured to be placed against a skin surface of a patient, for example, a wound dressing, a bed pad, a diaper, incontinence wear, or sanitary napkins. Absorbent article 100 includes a substrate 110 including a distal surface 113 and a proximal surface 117. Absorbent article 100 also includes an absorbent member 120 including a distal surface 123 in contact with at least portion of proximal surface 117 of substrate 110. In some examples, a center of absorbent member 120 may be aligned with a center of substrate 110. In some examples, absorbent member 120 can be sized to have a surface area that is less than the area of proximal surface 117 of the substrate 110.

In the examples shown in FIGS. 1 and 2., absorbent article 100 further includes an adherent member 130. Adherent member 130 defines a distal surface 133 and a proximal surface 137. In some examples, adherent member 130 is disposed adjacent proximal surface 117 of substrate 110. For example, distal surface 133 of adherent member 130 may contact at least a portion of proximal surface 117 of substrate 110. Optionally, adherent member 130 may be sized such that at least another portion of distal surface 133 of adherent member 130 can contact at least a portion of proximal surface 117 of substrate 110.

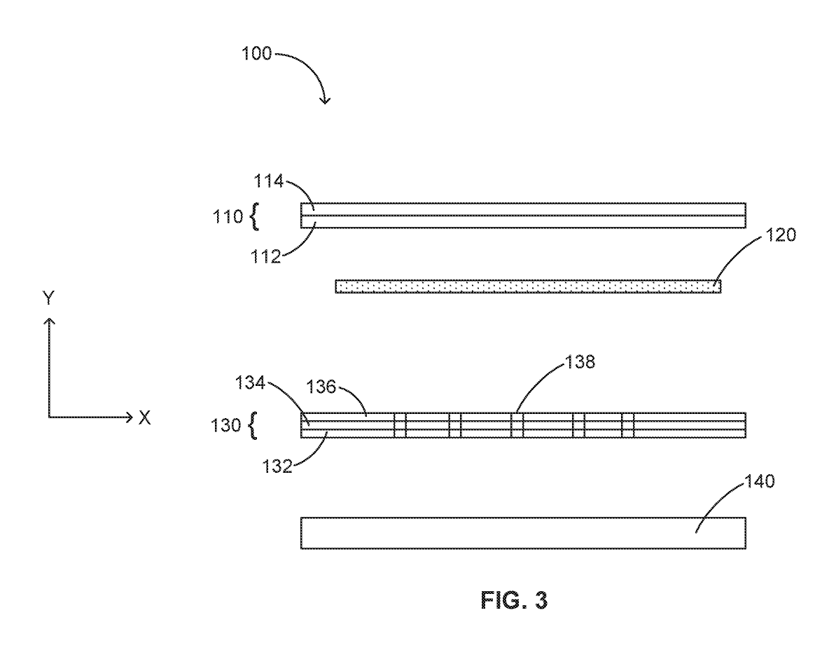

In the example shown in FIGS. 2 and 3, absorbent article 100 may further include at least one release member 140. FIG. 3 is a schematic and conceptual illustration showing an exploded cross-sectional view of absorbent article 100 of FIG. 2, with the cross-section passing though some perforations of plurality of perforations 138. Release member 140 may define a distal surface 143 in contact with proximal surface 137 of adherent member 130, and proximal surface 147. Release member 140 can be releasably removed from adhesive member 130 to expose adherent member 130 to a skin surface, for example, a portion of skin to which absorbent article 100 is to be secured or applied. Providing release member 140 may protect adherent member 130 from the external environment, and help in preventing contamination and maintaining adhesive properties of adherent member 130 up to the point at which absorbent article 100 is applied to a skin surface, for example, by removing release member 140 and contacting adherent member 130 to the skin surface.

Substrate 110 may serve as an outermost or sub-outermost member that is distal from the adherent layer 130 while also facilitating vapor permeability to accommodate body fluids or vapor, for example, from a skin surface to which absorbent article 100 is applied. In some examples, substrate 110 may facilitate vapor permeability to accommodate fluids or vapor, for example, perspiration, wound exudates, or other bodily fluids or vapors, from a skin surface adjacent a wound to which absorbent article 100 may be applied. The vapor permeability of substrate 110 may help reduce heat and moisture vapor build up within absorbent article 100 by allowing the moisture vapor to exit article 100 through substrate 110.

For example, absorbent article 100 may be applied to the skin surface such that adherent member 130 is proximal to and contacts the skin surface, while substrate 110 is distal from the skin surface. Thus, fluids from the skin surface may be absorbed across or through adherent member 130 (e.g., through perforations defined in adherent member 130) into absorbent member 120. Vapor generated from the skin, for example, by body heat, may permeate away from the skin surface through substrate 110, ultimately reducing accumulation of moisture in absorbent article 100. Such permeation may also provide cooling to skin surface in the vicinity of absorbent article 100, for example, by wicking away relatively warm perspiration from the skin surface. Thus, substrate 110 may include a moisture vapor permeable member or otherwise be moisture vapor permeable to facilitate vapor permeability through absorbent article 100. In some examples, substrate member 110 has a moisture vapor transmission rate of at least about 500 grams per meters squared per 24 hours (g/m²/day) under ambient conditions or at room temperature. A moisture vapor transmission rate of at least about 500 g/m²/day may help prevent accumulation or pooling of body fluids or exudates adjacent or under absorbent article 100. In some examples, substrate 110 may have a moisture vapor transmission rate greater than or equal to about 500 g/m²/day and less than or equal to about 5000 g/m²/day, for example, at least about 1000 g/m²/day.

In addition, in some examples, substrate 110 has a flexible configuration, and may facilitate conformability of absorbent article 100 when applied or adhered to curved or non-flat skin surfaces. In some examples, substrate 110 may serve as a supporting member having sufficient tensile strength to resist break or tearing in response to absorbent article 100 being removed from or applied on a skin surface.

Substrate 110 may include a liquid impermeable member, for example, to prevent or reduce migration of fluids into absorbent article 100, to prevent or reduce migration of fluids contained within absorbent member 120 outside of article 10, or both. Such migration of fluids into absorbent article 100 may result from inadvertent external contact with liquids, for example, during washing, cleaning, or bathing of the patient to which article 100 is applied. The liquid impermeable member may also be moisture vapor permeable, for example, allowing release of vapor away from a skin surface through absorbent article 100 while reducing flow of external fluid through absorbent article 100 toward the skin surface. To provide both liquid impermeability and vapor permeability, in some examples, substrate 110 may include a laminated member, as shown in FIG. 3.

In the example shown in FIG. 3, substrate 110 includes a moisture vapor permeable laminate including a first layer 112 and a second layer 114 that is contiguous with and in contact with a distal surface of first layer 112. In some examples, substrate 110 can consist essentially of first layer 112 and second layer 114 that is contiguous with and in contact with first layer 112.

First layer 112 may be formed from any suitable material. For example, first layer 112 may include a breathable film, for example, as a vapor permeable polyethylene film. Second layer 114 may also be formed of any suitable material, and can be the same as or different than first layer 112. For example, second layer 114 may include a non-woven fabric, for example, a spun bond polypropylene material. The non-woven fabric of second layer 114 may be secured to first layer 112 by, for example, heat bonding, adhesive bonding, or static bonding. In some examples, securing first and second layers 112 and 114 together may be facilitated by corona treatment of the facing surface of one or both of first and second layers 112 and 114.

Absorbent member 120 may be formed from any suitable material that is configured to absorb and substantially retain fluids that contact a surface of absorbent article 100, for example, fluid exudates or respiration. For example, absorbent member 120 may include absorbent material. In some examples, absorbent member 120 may include superabsorbent material, for example, in addition to, or instead of, a non-superabsorbent material. For example, the superabsorbent material may include superabsorbent particles (SAP), disposed, e.g., embedded, in a cellulosic matrix, such as tissue layers. Superabsorbent material is material that is capable of absorbing fluid several times its own volume. The use of superabsorbent material may allow absorbent member 120 to be relatively compact, for example, relatively thin in a direction across a thickness of absorbent article 100, for example, in a direction along the y-axis shown in FIGS. 2 and 3 (orthogonal x-y-z axes are shown in the figures for ease of description only), or relatively occupying a lower area relative to an area of absorbent article 100. For example, absorbent article 100 including superabsorbent material in absorbent member 120 may exhibit a low profile, avoiding problems associated with high profile wound dressings, for example, pressure ulcers. Thus, absorbent member 120 may include superabsorbent material such as SAP disposed within tissue layers that may be bonded together by an adhesive, by use of a latex sprayed thereon, or by effecting hydrogen bonding under application of high temperature and pressure onto the tissue layers. In some examples, absorbent member 120 may further include fluff pulp material in addition to or instead of the SAP. For example, absorbent member 120 may include SAP and fluff pulp disposed within tissue layers. In some examples, absorbent member 120 may consist essentially of SAP, fluff pulp, and a cellulosic matrix. In other specific configurations, absorbent member 120 can consist essentially of SAP and a cellulosic matrix.

In some examples, absorbent member 120 may include superabsorbent material, such as superabsorbent fibers, in a matrix of synthetic fibers. For example, absorbent member 120 may include superabsorbent fibers disposed within a matrix of polyester fibers that are needle-punched to form a nonwoven mat. In some examples, absorbent member 120 may further include fluff pulp material. For example, absorbent member 120 may include superabsorbent fibers, fluff pulp and polyester fibers needle-punched into a nonwoven layer. In some examples, absorbent member 120 may consist essentially of a needle-punched layer of superabsorbent fibers, fluff pulp, and polyester fibers. In other examples, absorbent member 120 may consist essentially of a needle-punched layer of superabsorbent fibers and polyester fibers.

While absorbent article 100 may include a unitary or single absorbent member 120, absorbent article 100 may include more than one absorbent member 120, for example, a plurality of absorbent members that may be spaced apart to allow for uniform transport and retention of fluid within absorbent article 100.

In some examples, adherent member 130 includes an adhesive for adhering absorbent article 100 to a skin surface. The adhesive may include, for example, a biologically inert adhesive composition. Adherent member 130 may include or consist essentially of a non-laminated structure, e.g., a single layer, for example, as shown in FIG. 1, adhesively secured to absorbent member 120 and, optionally, to substrate 110. For example, adherent member 130 may consist essentially of a single silicone layer. Thus, adherent member 130, whether consisting essentially of a non-laminated structure or including a laminated structure, may be applied on or secured to at least a portion of a surface of absorbent member 120, and, optionally, to at least a portion of a surface of substrate 110, for example, on a portion of a proximal surface of first layer 112 of substrate 110. In some examples, absorbent article 100 may be assembled by spraying or brushing an adhesive composition onto one or more interfacing or contacting surfaces of any one or more of substrate 110, absorbent member 120, and adherent member 130.

In other examples, adherent member 130 may include a plurality of laminated layers, for example, a first adhesive layer 132, an adhesive support layer 134, and a second adhesive layer 136, as shown in FIG. 3. First adhesive layer 132 may be configured to adhere to a skin surface, may include any suitable adhesive, for example, a biologically inert adhesive composition. In some examples, the adhesive may include a silicone, e.g., a silicone-based adhesive. The adhesive of first adhesive layer 132 may, for example, consist essentially of the silicone-based adhesive. Second adhesive layer 136 may include any adhesive suitable for adhering adherent member 130 to one or both of absorbent member 120 or substrate 110. In some examples, the adhesive may include a pressure sensitive adhesive. For example, second adhesive layer 136 may include or consist essentially of an acrylic adhesive. Adhesive support layer 134 may include a material on which one or both of first adhesive layer 132 and second adhesive layer 136 may be applied or supported. For example, adhesive support layer 134 may include a polymer film, a polyurethane film. In other examples, however, first and second adhesive layers 132, 136 may be directly adhered or otherwise connected together.

In some examples, adherent member 130 defines a plurality of perforations 138 through a thickness thereof, for example, in a direction along the y-axis, as shown in FIG. 3. For example, plurality of perforations 138 may extend through a layer of silicone. Plurality of perforations 138 may also extend through one or more layers that may be present in adherent member 130. A silicone layer without perforations may note allow sufficient flow or transport of fluids or other substances through adherent member 130 to absorbent member 120. For example, a bulk of the silicone layer may be liquid impermeable. Plurality of perforations 138 may one or more of facilitate skin respiration, wound exudate transport from a wound site to absorbent member 120, or vapor permeability through absorbent article 100. Without wishing to be bound by theory, the size, shape, and distribution of the plurality of perforations 138 may control the rate of wound exudate absorption and the vapor permeability of absorbent article 100.

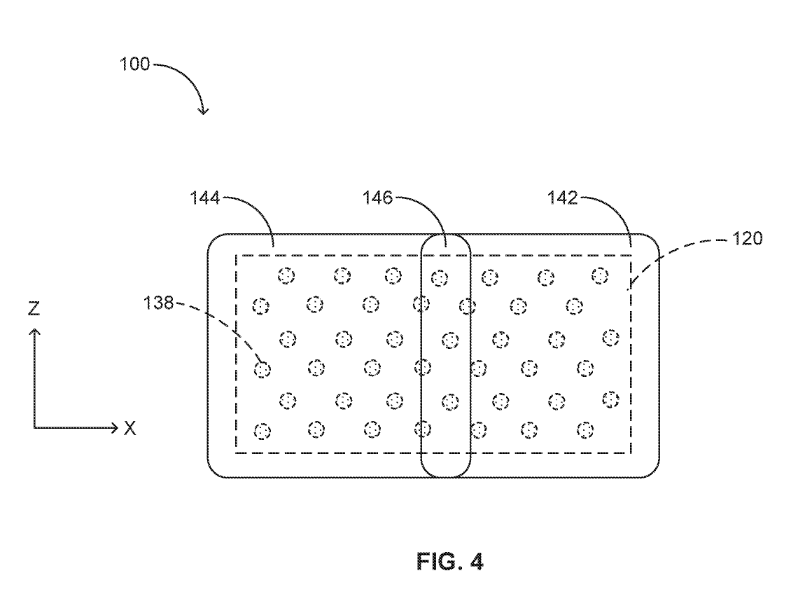

Perforations 138 may have any suitable size and shape for facilitating the conveyance of fluids through adherent member 130 to absorbent member 120, while still allowing adherent member 130 to help substantially fix (e.g., maintain one position or move a negligible amount relative to the wound site or other target site on the patient to allow article 100 to cover the target site) absorbent article 100 in place on a skin surface of a patient. In some examples, plurality of perforations 138 may include perforations defining one or more of circular, square, triangular, elliptical, rectangular, polygonal, or any other closed boundaries. Plurality of perforations 138 may having an average diameter between about 0.1 millimeters (mm) and about 1 mm, such as about 0.635 mm (25 thousandths of an inch), measured in a direction perpendicular to the thickness of member 130 (in an x-z plane, shown in FIGS. 2 and 4). Each perforation of the plurality of perforations 138 may have substantially the same diameter, accounting for manufacturing tolerances. In other examples, the plurality of perforations 138 may include perforations having a non-uniform range of diameters with an average diameter between about 0.1 mm and about 1 mm. If the average diameter of each of the perforations 138 is too small, then the flow of fluids or exudates across a thickness of adherent member 130 into absorbent member 120 may be impeded. On the other hand, if the average diameter of each of the perforations 138 is too large, then drying fluids or exudates may induce adherence to a skin surface of a patient and cause adherent member 130 to become adherent. In some examples, adherent member 130 may define at least about 300 holes per square inch (at least about 45 holes per square centimeter), for example, about 370 holes per square inch.

To promote uniform transport of fluids or exudates across adherent member 130 into absorbent member 120, plurality of perforations 138 may include perforations uniformly distributed throughout adherent member 130. In other examples, a higher density of uniformly distributed perforations of larger size, e.g., larger diameter perforations, may be disposed at region of adherent member 130 overlaying absorbent member 120 compared to smaller diameters perforations overlay only substrate 110. In further examples, a lower density or even no perforations may be disposed at certain areas of the adherent member 130, e.g., at areas overlaying substrate 110. In some examples, the size and density of plurality of perforations 138 may provide an aggregate exposure area in a range of from about 10% to about 30%, e.g., about 15%, of the surface area of a surface area of adherent member 130. To help prevent or reduce penetration or movement of portions of absorbent member 120 through adherent member 130, the size and density of plurality of perforations 138 may be maintained so that a sufficiently small exposure area is provided to absorbent member 120.

Release member 140, shown in FIGS. 2-4, may protect the adhesive surface of adhesive member 130, and may further provide rigidity to absorbent article 100 prior to use or application onto a skin surface. Release member 140 may include a polymer film that has a lower adhesion affinity to adherent member 130, for example, to the first adhesive layer 132, compared to the adhesion affinity between first adhesive layer 132 and substrate 110, or absorbent member 120, or to adhesive support layer 134, as well as compared to the adhesion affinity of second adhesive layer 136 to adhesion support layer 134, to substrate 110, or to absorbent member 120. This may allow release member 140 to be released from absorbent article 100 without moving or affecting the structure or arrangement of other layers or members of absorbent article 100.

Release member 140 may have a sufficient thickness, measured along the y-axis shown in FIGS. 2 and 3 (orthogonal x-y-z axes are shown in the figures for ease of description only), to provide a desired stiffness or rigidity to absorbent article 100. For example, release member 140 can comprise a polycarbonate film to provide stiffness. In some examples, as illustrated in FIG. 3, release member 140 includes a unitary film. In other examples, as illustrated in FIG. 4, release member 140 may have multiple components including a first releasing portion 142 and a second releasing portion 144, optionally with an overlapping or portion 146 that covers or overlays over a portion of first releasing portion 142 which facilitates removal, e.g., release, from and exposes adhesive member 130 for adhesion to the skin surface. In some examples, the thickness of release member 140, or one or more portions thereof, may be in a range of from about 0.05 mm (about 2 mil) to about 0.127 mm (about 56 mil). To allow for relatively easy peeling off of release member 140 from adherent layer 130 prior to the point of application of absorbent article 100, release member 140 may include unsecured portions (not shown), e.g., flaps, at one or more ends of each unsecured or releasing portion to facilitate removal or release of such portions from the rest of absorbent article 100.

Example techniques according to the disclosure may be used to prepare (e.g., form or manufacture) example absorbent articles, for example, those described with reference to FIGS. 1 to 4. An example technique for forming absorbent article 100 may include securing absorbent member 120 to substrate 110, and securing at least a portion of adherent member 130 comprising silicone to at least a portion of absorbent member 120. In some examples, the example technique may further include securing at least another portion of adherent member 130 to at least a portion of substrate 110. In some examples, one or both of the securing absorbent member 120 or securing at least a portion of adherent member 130 includes one or more of heat bonding, adhesive bonding, static bonding, or corona treatment.

In some examples, the example technique further includes laminating second layer 114, for example, including a nonwoven layer, first layer 112, for example, a supporting film, to form substrate 110. While these example techniques may be used to prepare the example absorbent articles discussed with reference to FIGS. 1 to 4, example techniques according to the disclosure may also be used to prepare other example absorbent articles.

Thus, in some examples, example absorbent articles may include an adherent member including silicone configured to contact a skin surface, for example, a surface having a wound. The silicone may be of medical grade. The adherent member including silicone may define perforations, which may allow wound fluids or other secretions to pass through the adherent member and or otherwise get absorbed by the absorbent member of the absorbent article, while continuing to secure absorbent article 100 to the skin surface.

Various examples have been described. These and other examples are within the scope of the following claims.

What is claimed:

1. An absorbent article comprising:
an adherent member comprising silicone, wherein the adherent member defines a plurality of perforations extending through a thickness of the adherent member;
a substrate adjacent the adherent member; and
an absorbent member disposed between the adherent member and the substrate.

2. The absorbent article of claim 1, wherein the adherent member is configured to adhere to skin of a patient.

3. The absorbent article of c aim 1, wherein the adherent member comprises a plurality of laminated layers.

4. The absorbent article of claim 3, wherein at least one layer of the plurality of laminated layers consists essentially of a silicone layer configured to contact skin of a patient and adhere the absorbent article to skin.

5. The absorbent article of claim 3, wherein at least one layer of the plurality of layers consists essentially of an adhesive layer adhering the absorbent member to the adherent member.

6. The absorbent article of claim 1, further comprising a release member disposed on the adherent member.

7. The absorbent article of claim 6, wherein the release member comprises a polycarbonate film.

8. The absorbent article of claim 1, wherein the absorbent member comprises a superabsorbent material.

9. The absorbent article of claim 1, wherein the substrate is moisture vapor permeable.

10. The absorbent article of claim 9, wherein the substrate has a moisture vapor transmission rate of at least about 500 grams per meters squared per day.

11. The absorbent article of claim 9, wherein the substrate comprises a laminated member.

12. The absorbent article of claim 11, wherein the laminated member comprises a nonwoven layer and a supporting film.

13. A wound dressing comprising absorbent article of claim 1.

14. A method of forming an absorbent article, the method comprising:
securing an absorbent member to a substrate; and
securing at least a portion of an adherent member comprising silicone to at least a portion of the absorbent member, wherein the adherent member defines a plurality of perforations extending through a thickness of the adherent member.

15. The method of claim 14, further comprising securing at least another portion of the adherent member to at least a portion of the substrate.

16. The method of claim 14, further comprising applying a release member on the adherent member.

17. The method of claim 14, wherein the securing comprises one or more of heat bonding, adhesive bonding, static bonding, or corona treatment.

18. The method of claim 14, wherein the absorbent member comprises a superabsorbent material.

19. The method of claim 14, wherein the substrate is moisture vapor permeable.

20. The absorbent article of claim 19, wherein the substrate has a moisture vapor transmission rate of at least about 500 grams per meters squared per day.
